(19) United States
(12) Patent Application Publication
(10) Pub. No.: US 2017/0303864 A1
(43) Pub. Date: Oct. 26, 2017

(54) MOTION SENSING DEVICE

(71) Applicant: Tina Su, Taichung City (TW)

(72) Inventor: Tina Su, Taichung City (TW)

(21) Appl. No.: 15/136,977

(22) Filed: Apr. 24, 2016

(51) Int. Cl. A61B 5/00; A61B 5/11

(52) U.S. Cl. CPC A61B 5/742; A61B 5/746; A61B 5/0015; A61B 5/11; A61B 5/6804

(57) ABSTRACT

A motion sensing device comprises a fixture, a display member, a sensing member, and a mobile member. The fixture is arranged with an user. T display member is assembled to an outer surface of the fixture. The sensing member is assembled to the fixture for sensing a physiological condition of the user. The sensing member includes a control unit and a transmission unit. The control unit is electrically connected to the display member. The sensing member includes a power supply unit for driving the display member. The mobile member is electrically and wirelessly connected to the sensing member and includes a receiving unit which is wirelessly connected to the transmission unit. The physiological condition of the user may be shown on the display member through the sensing member and then wirelessly transmitted to the mobile member for providing to others or himself/herself to know anytime and anywhere.

### BACKGROUND OF THE INVENTION

### 1. Field of the Invention

The present invention relates to a motion sensing device, and especially relates to a motion sensing device with clothes capable for being worn on an user and wirelessly connecting to a mobile member or an outer transmission device. A display member arranged on the clothes may show/display the message(s) with alarm, words, or figures. It is convenient for other to know and recognize what the condition of the user is.

### 2. Description of Related Art

For people, clothes is not only covering the body but also shielding wind and blocking sun to prevent the body from exposing outside and hurting or infecting by the foreign body. Besides, it is fashionable and beautiful. The clothes is more and more diversified along with time change and progress. In addition, clothes is a dispensable part, lots of people also care the quality of life. The quality of life is not only a superior life but also the life of health and safety. The life of health is mainly focused on eating and moving. The life of health may be achieved by eating healthily and move truly. The life of safety may be achieved by taking care of family and many instruments are also needed. There are some monitors arranged in the house which cooperate with the smart phones to control and monitor so as to make sure the safety of the family (old people and children). However, the blind visions of the monitors may be existed. It is hard to follow the movement of the old people or children to change the positions. The drawbacks are existed.

In order to solve the problems/drawbacks as above mentioned, please reference to Taiwanese publication patent no. 201031351. It disclosed that an exercise/health garment capable of measuring physiological state to provide user with a wearing, which includes two sensing parts, a conductive garment and a measurement device. The conductive garment is equipped with two conductive cloths at appropriate position as a signal transmission media. Each of the two sensing parts has a sensing electrode electrically connecting to conductive cloth. The measurement device is equipped on the conductive garment to electrically connect with said two conductive cloths. Two sensing parts provide user with a wearing so that said two sensing electrodes can contact two measurement points on the user's body for acquiring user's physiological signals. The measurement device can measure user's physiological state by using two sensing electrodes to acquire user's physiological signals and by using conductive cloths on conductive garment to do a conduction.

The exercise/health garment as above mentioned may remind the user to alarm the physical changes so that the user may know is/her own conditions. But some drawbacks are still existed and described as follows.

Firstly, although the connection of the sensing electrode and the exercise/health garment may alarm, the alarm is performed only by voice so as not to understand what the physical conditions of the user are immediately. If misjudging, it will make troubles to others.

Secondly, it only provides the conditions to the user himself/herself to know but others do not. It may delay the time of making emergency treatment/first aid.

Thirdly, the structure is complicated and it may be activated and used while the electronic elements are electrically connected with one another.

In view of the foregoing circumstances, the inventor has invested a lot of time to study the relevant knowledge, compare the pros and cons, research and develop related products. After quite many experiments and tests, the “motion sensing device” of this invention is eventually launched to improve the foregoing shortcomings, to meet the public use.

### SUMMARY OF THE INVENTION

A main object of this invention is providing a motion sensing device. It may be arranged on the clothes which may be worn on an user for alarming. Besides the user may know the physiological condition himself/herself, others around the user may be also know immediately what the condition of the user is when the accident happens. A second object of the present invention is that it may be assembled to the clothes in a easiest manner to achieve the effects of precise measurement and alarm.

In order to achieve above mentioned objects, a first embodiment of a motion sensing device comprises a fixture, arranged with an user; a display member, assembled to an outer surface of the fixture; a sensing member, assembled to the fixture for sensing a physiological condition of the user, the sensing member includes a control unit and a transmission unit which are built therein, the control unit is electrically connected to the display member, and further the sensing member includes a power supply unit for driving the operation of the display member; and a mobile member, electrically and wirelessly connected to the sensing member, the mobile member includes a receiving unit which is built therein, and the receiving unit is wirelessly connected to the transmission unit built-in the sensing member; wherein the physiological condition of the user may be shown on the display member through the sensing member assembled to the fixture and cooperated with the display member, and then the physiological condition is wirelessly transmitted to the mobile member for providing to others or himself/herself to know anytime and anywhere.

Preferably, in the first embodiment, the display member is a flexible liquid crystal display, a button, or a light strip.

Preferably, in the first embodiment, the display member is the button and a light emitting element is arranged therein, the light emitting element is electrically connected to the sensing member, and the power supply unit is driving the light emitting element to illuminate.

Preferably, in the first embodiment, the control unit is built-in a flash mode, an alarm mode, a safety mode, and a race mode.

Preferably, in the first embodiment, the mobile member is a smart phone, a pad or tablet, or a transmitter, the connection of the mobile member and the sensing member is with cloud, bluetooth, infrared rays, or radio frequency.

Preferably, in the first embodiment, the fixture is clothes, a waistband, a wristband, an armband, or a sports bra.

In order to achieve above mentioned objects, a second embodiment of a motion sensing device comprises a fixture, worn on an user and having at least one sewing portion; a display member, assembled to an outer surface of the fixture; a sensing member, assembled to the fixture for sensing a physiological condition of the user, the sensing member includes a control unit and a transmission unit which are built therein, the control unit is electrically connected to the display member, and further the sensing member includes a power supply unit for driving the operation of the display member; and at least one lead is built in the at least one sewing portion, two ends of the at least one lead are respectively electrically connected to the display member and the sensing member; wherein the physiological condition of the user may be shown on the display member through the sensing member assembled to the fixture and cooperated with the display member, and then the physiological condition is wirelessly transmitted to the mobile member for providing to others or himself/herself to know anytime and anywhere; and wherein the at least one lead is built-in the at least one sewing portion so as to match a body curve of the user.

Preferably, in the second embodiment, the display member is a flexible liquid crystal display, a button, or a light strip; the display member further includes a reader built therein, the reader is read a portable external hard driver, a SIM card, or an USB flash drive and transmitted data to the sensing member, the sensing member includes a receiving unit which is electrically connected to the control unit, and the receiving unit is received the data read from the reader.

Preferably, in the second embodiment, the display member is the button and a light emitting element is arranged therein, the light emitting element is electrically connected to the sensing member, and the power supply unit is driving the light emitting element to illuminate.

Preferably, in the second embodiment, the control unit is built-in a flash mode, an alarm mode, a safety mode, and a race mode.

Preferably, in the second embodiment, the fixture is clothes, a girdle, a wristband, an armband, or a sports bra.

In summary, the advantage of the present invention is that when it is worn on the user, the sensing member may sense the change of the physiological condition of the user. When the condition is abnormal, the control unit built-in the sensing member may immediately control the display member to operate, and simultaneously the transmission unit built-in the sensing member may transmit the abnormal message/data/signal to the receiving unit built-in the mobile member. Therefore, the user and the people around the user may immediately know what the condition of the user is and further make appropriate treatment through the display member. The people who hold the mobile member may immediately know what the abnormal condition of the user is and do relative operations to reduce and prevent from the dangers. Besides, for the second embodiment, the lead is buried in the fixture so as to make it neat and beautiful. And further, the lead may be buried in the sewing portion to correspond to the body curve of the user so as to make the user comfortable. Besides it may keep the sensing function, it may further make the user comfortable while wearing to move or exercise.

The various objects and advantages of the present invention will be more readily understood from the following detailed description when read in conjunction with the appended drawing.

### BRIEF DESCRIPTION OF THE DRAWINGS

FIG. 1 is a block diagram of a first embodiment of a motion sensing device of the present invention;

FIG. 2 is an operational view of a display member shown a relative data to a mobile member of the first embodiment of the present invention while a user worn a fixture is tumbled down or occurred an accident;

FIG. 3 is a view of the motion sensing device of the present invention while racing;

FIG. 4 is an operational view of the motion sensing device of the present invention while the fixture is an armband;

FIG. 5 is an operational view of the motion sensing device of the present invention while the fixture is a sports bra;

FIG. 6 is a perspective view of the motion sensing device of FIG. 5 viewed from another angle;

FIG. 7 is a block diagram of a second embodiment of a motion sensing device of the present invention;

FIG. 8 is a perspective view of the second embodiment of the motion sensing device of the present invention while the fixture is clothes with sewing portions;

FIG. 9 is a view of the motion sensing device of the present invention while the power of the display member is shortage to be in a flash mode; and

FIG. 10 is a view of the motion sensing device of the present invention while the display member is connected with a SIM card.

### DETAILED DESCRIPTION OF THE INVENTION

To describe clearly that the present invention achieves the foregoing object and function, the technical features and desired function are described with reference to a preferred embodiment and accompanying drawings.

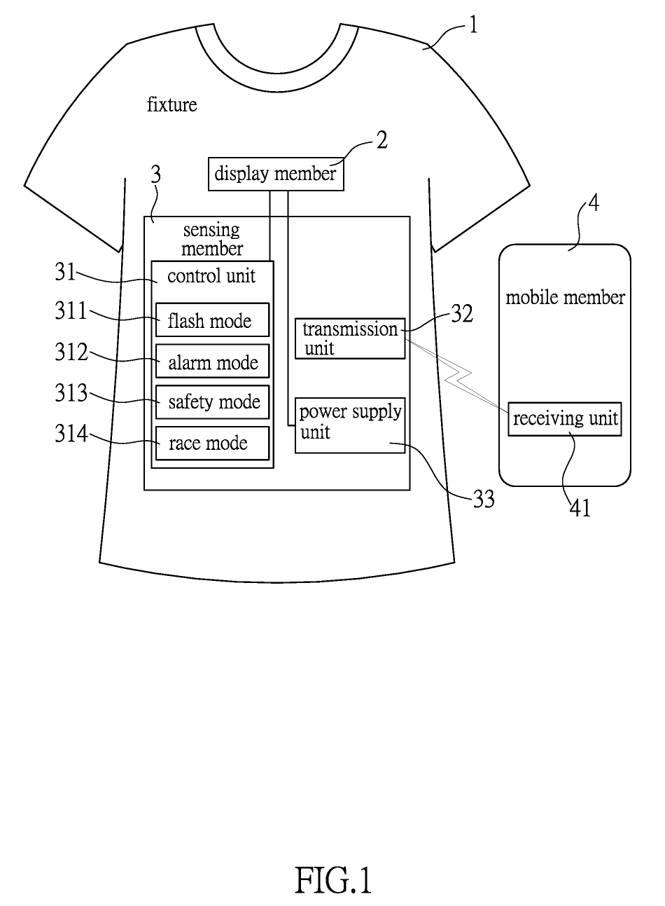

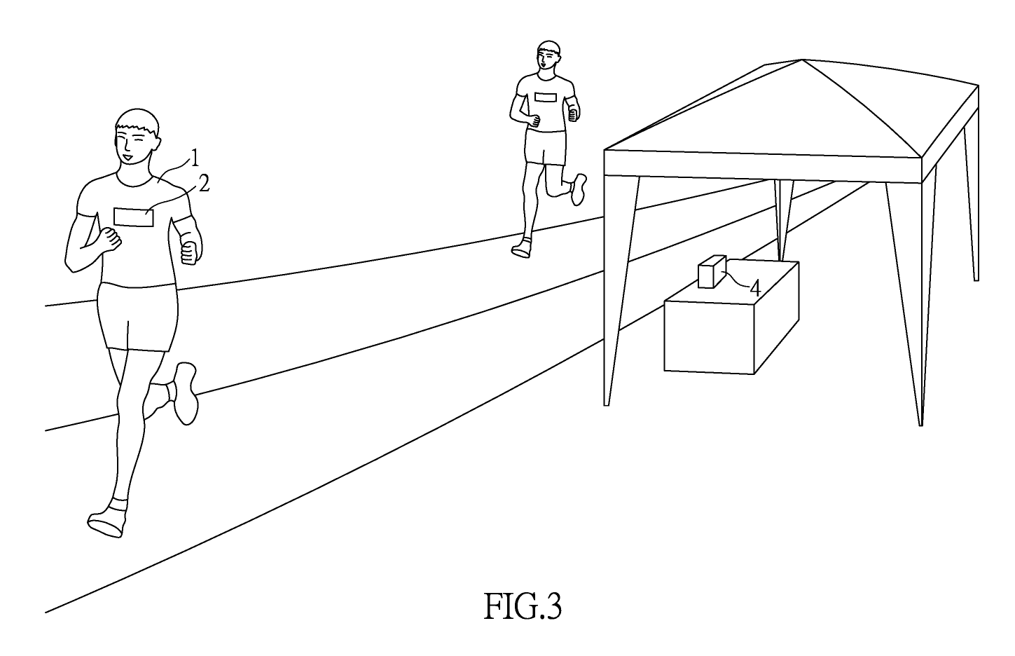

The motion sensing device of the present invention has two embodiments. A first embodiment is described as follows firstly. Please reference to FIGS. 1 to 6, a motion sensing device may comprise a fixture 1, a display member 2, a sensing member 3, and a mobile member 4. The fixture 1 is provided for wearing on a user. The display member 2 is assembled to an outer surface of the fixture 1 and shown data to others or himself/herself to see and know a physiological condition of the user. The sensing member 3 is used for sensing the physiological condition of the user worn the fixture 1. The sensing member 3 is assembled to the fixture 1 and sensed the physiological condition of the user. The sensing member 3 may be arranged at an inner surface of the fixture 1 to contact with the skin of the user. Otherwise, the sensing member 3 may be detachably arranged at the outer surface of the fixture 1. It is convenient for the user to disassemble the sensing member 3 and clean/wash the fixture 1. The sensing member 3 further includes a control unit 31 and a transmission unit 32 which are built therein. The control unit 31 is electrically connected to the display member 2. The sensing member 3 further includes a power supply unit 33 for driving the operation of the display member 2. The mobile member 4 is wirelessly and electrically connected to the sensing member 3. The mobile member 4 may include a receiving unit 41 which is built therein. The receiving unit 41 is wirelessly connected to the transmission unit 32 which is built-in the sensing member 32. The physiological condition of the user may be shown on the display member 2 through the sensing member 3 assembled to the fixture 1 and cooperated with the display member 2. And then, the physiological condition is wirelessly transmitted to the mobile member 4 for providing to others or himself/herself to know anytime and anywhere so as to achieve the effects of safety and convenience. (The above mentioned embodiment is the main skill feature of this invention and corresponds to the claim 1 of this invention to understand the object and embodiments of this invention in detail. And the skill features of the depending claims are for describing the claim 1 in detail or adding more skill features, but not limited thereto. It should be known that the claim 1 is not necessary to include the skill features of the depending claims.)

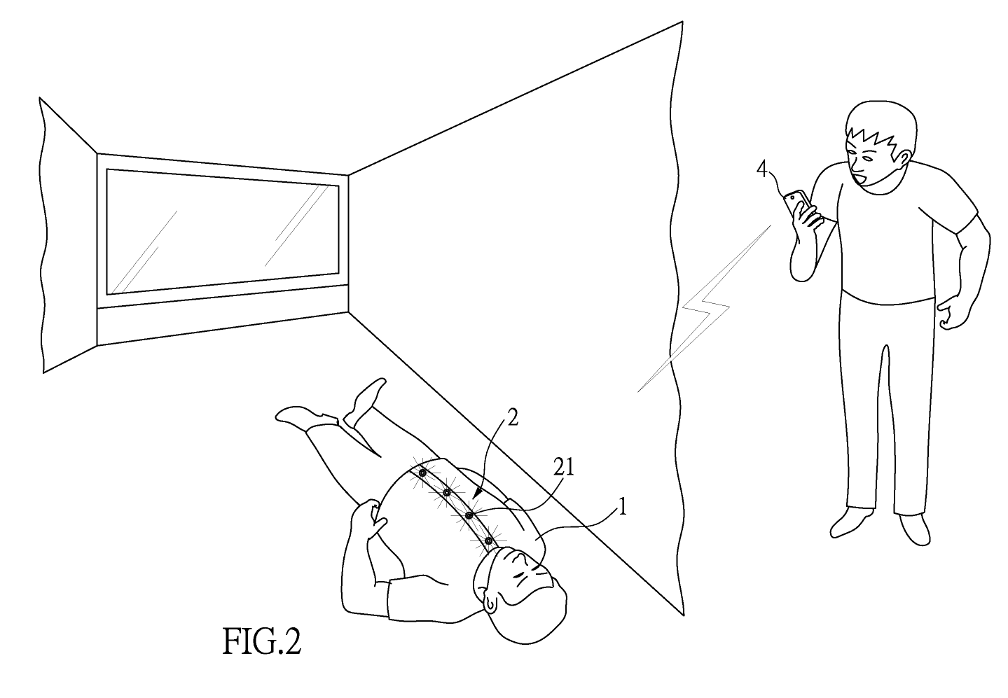

According to above descriptions, the main function of the motion sensing device of the present invention is providing the people with disabilities to wear. The people with disabilities may be children, old people, or patients. When the fixture 1 of the motion sensing device of the present invention is worn on the user, the sensing member 3 assembled to the fixture 1 may sense the physiological condition of the user, such as heart beats, blood pressure, and body temperature. When the physiological condition sensed by the sensing member 3 is abnormal, an abnormal message (maybe a data) is firstly shown on the display member 2. Besides it may provide to be known by himself/herself, others around the user may also know what happened immediately so as to make emergency treatment. In addition, at the same time, because the transmission unit 32 is built-in the sensing member 3, the sensing member 3 may wirelessly transmit the abnormal message (or data) to the mobile member 4 which is held on someone's hand with the transmission unit 32. After the abnormal message (or data) is received by the mobile member 4 with the receiving unit 41, the relative connections an treatments may be processed immediately. The dangers of the user while accidents are happened may be reduced and the emergency care personnel may make appropriate emergency treatments through the two different kinds of message transmission (showing message/data on the display member 2 and wirelessly transmitting message/data to the mobile member 4) (shown as in FIG. 2).

The display member 2 may be a flexible liquid crystal display (LCD), a button, or a light strip, but not limited thereto. The flexible LCD is like a screen which is capable for showing the message/data with relative words and figures. The display member 3 of the first embodiment of the present invention is illustrated by the button for example. Please refer to FIGS. 1 and 2, when the heartbeat of the user is accelerated and the user is hard to stand to tumble down and the sensing member 3 is sensed the heartbeat rate is higher than the general rate range, the message/data may be shown on the display member 3 to alarm because the sensing member 3 is sensing anytime. When the display member 3 is the button, a light emitting element 21 is further arranged in the button. The light emitting element 21 is electrically connected to the sensing member 3 and is driven to illuminate y the power supply unit 33. That is, when the condition is abnormal, the display member 2 is controlled to activate by the control unit 31 after the abnormal condition is sensed by the sensing member 3. For example, the light emitting element 21 may be controlled by the control unit 31 of the sensing member 3 to illuminate. When the light emitting element 21 is illuminated abnormally, the physiological condition of the user is judged to be abnormal so as to make treatments immediately. It may shorten the golden first aid. When the display member 2 is the flexible LCD, the relative words and figures may be shown thereon to provide the people around the user to know what the condition is (not shown). No matter what the display member 2 is the button, the light strip, or the flexible LCD, the main object of the motion sensing device of the present invention is providing himself/herself and people around the user to immediately know what the condition of the user is and then quickly make treatments.

Following above description, when the physiological condition of the user worn the fixture 1 is abnormal, in order to provide the user himself/herself and people around the user to clearly recognize if the abnormal condition is happened, the control unit 31 includes various modes (built therein) to change the showing of the display member 2. The modes may include a flash mode 311, an alarm mode 312, a safety mode 313, and a race mode 314. Please refer to FIG. 1, the flash mode 311 means that the display member 2 is flashed quickly to make the people around the user and the emergency care personnel to know what the condition is when the physiological condition of the user is abnormal. The alarm mode 312 means that the display member 2 may prompt (the light emitting element 21 of the button is lighted with orange rays, the flexible LCD shows words, figures, and light colors to prompt) when the physiological condition of the user starts abnormal changes. The safety mode 313 means that the user may know the physiological condition of the user is stable and normal right now (the modes mentioned above are not limited thereto, they may be added or subtracted according to the demand of the user).

Furthermore, please refer to FIGS. 1 and 2, when the physiological condition sensed by the sensing member 3 is abnormal, the display member 2 is controlled by the control unit 31 to operate At the same time, an abnormal signal is transmitted by the transmission unit 32 built-in the sensing member 3. That is, when the physiological condition of the user is abnormal, the abnormal signal is generated by the sensing member 3 to store in the transmission unit 32 and then the abnormal signal is transmitted from the transmission unit 32 to the receiving unit 41 built-in the mobile member 4. The mobile member 4 may be a smart phone, a pad/tablet, or a transmitter (but not limited thereto). The connection of the mobile member 4 and the sensing member 3 bay be with cloud, bluetooth, infrared rays, or radio frequency. The mobile member 4 may be also worn by the user himself/herself or others, such as relatives and friends, sons and daughters, parents, or caregivers. Therefore, the abnormal signal sensed by the sensing member 3 may be immediately known by the people who do not stay around the user.

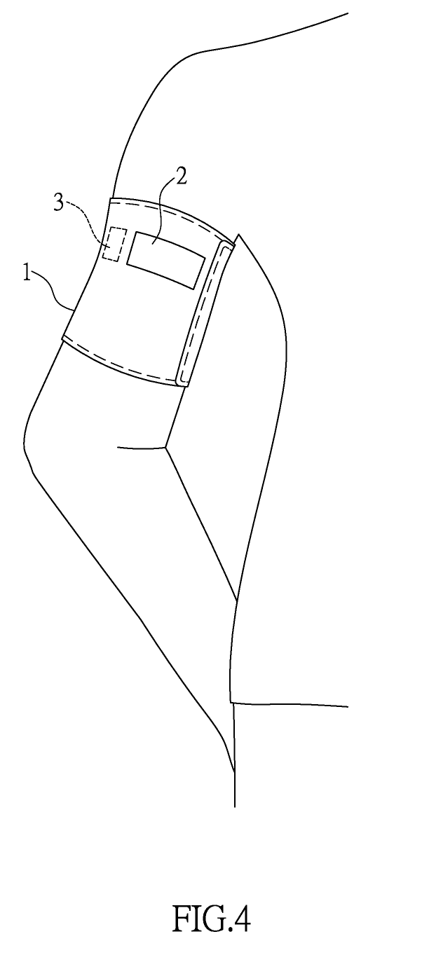

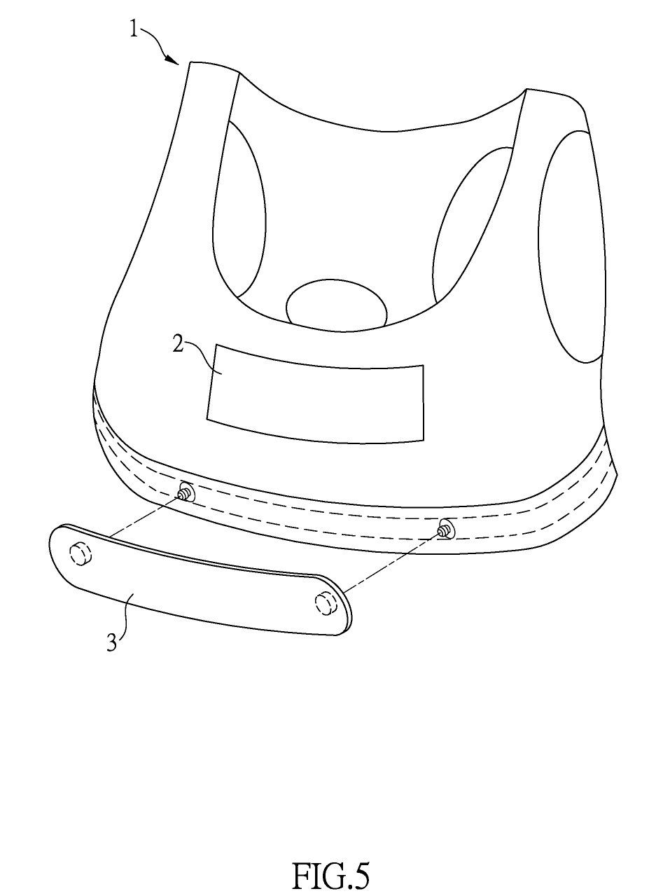

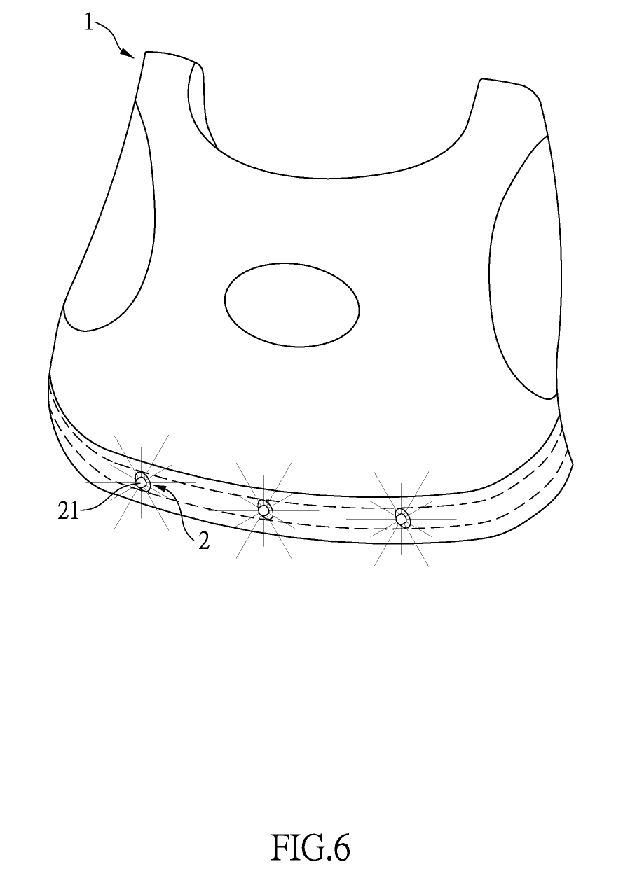

The motion sensing device of the present invention is not only applied to the emergency technical field but also competitions, such as racing, beating, and gaining. For example, please refer to FIGS. 1 and 4, when lots of road runners wear the fixture 1 and the competition starts, the display member 2 (button) arranged on the corresponding road runner may show a first color which is orange for example and refers to a first lap. When the first lap is finished and a second lap starts at the start, the mobile member 4 (the transmitter is preferred) is arranged at the start. When the road runner passes through a sensing range of the mobile member 4, the orange color of the display member 2 may be changed by the mobile member 4 so as to let the sponsor know the road runners who start the second lap and further distinguish the condition of each road runner for the recorder convenient to record and recognize. The mobile member 4 may simultaneously record the physiological condition of the runner himself/herself so as to provide the sponsor to keep eyes on. The fields of the motion sensing device of the present invention may be applied widely. The motion sensing device of the present invention may be used to the sports competitions for clear recognition. In addition, The fixture 1 may be clothes, a waistband, a wristband, or an armband. It is mainly capable for providing the sensing member 3 to truly sense the physiological condition of the user and show/display the relative messages/data so as to achieve the effect of emergency treatment(first aid) or convenience others to recognize. Please refer to FIG. 4, the fixture 1 is the armband tied on the arm of the user. The sensing member 3 may be also applied to a sports bra so as to provide the user to wear. The sensing member 3 arranged on the sports bra may be detached by the user himself/herself. It is convenient for the user to detach/disassemble the sensing member 3 from the fixture 1 and then clean the fixture 1. The design of the sensing member 3 of other embodiments may be detachable (shown as in FIG. 5). FIG. 6 is the back view of FIG. 5. The display member 2 having the light emitting element 21 may be further arranged at the back of the sports bra to provide others who stay around the user in different positions to clearly recognize.

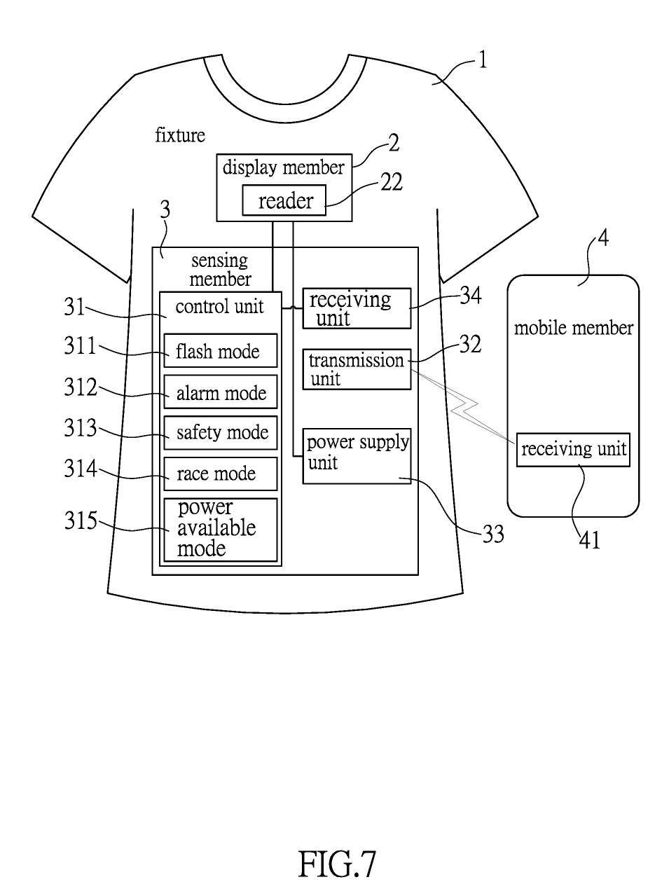

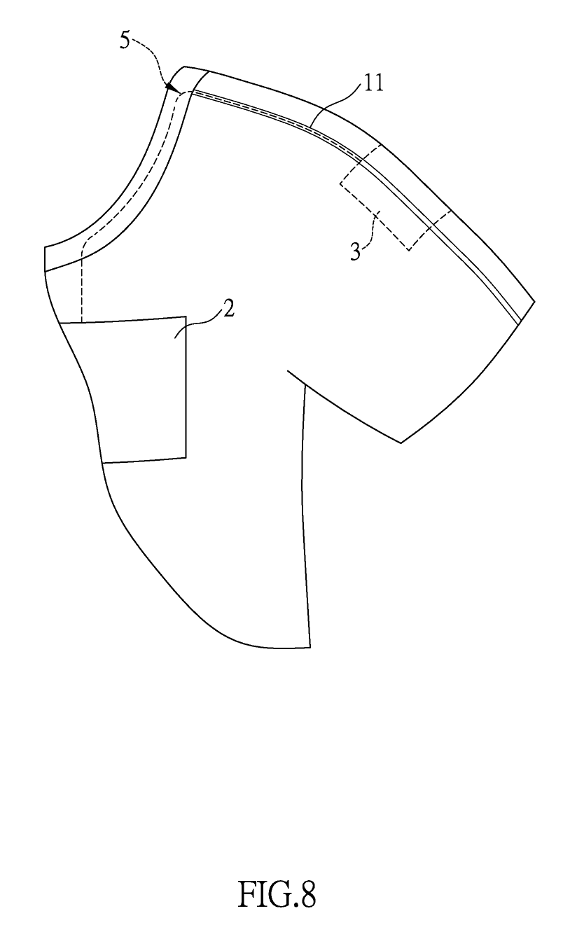

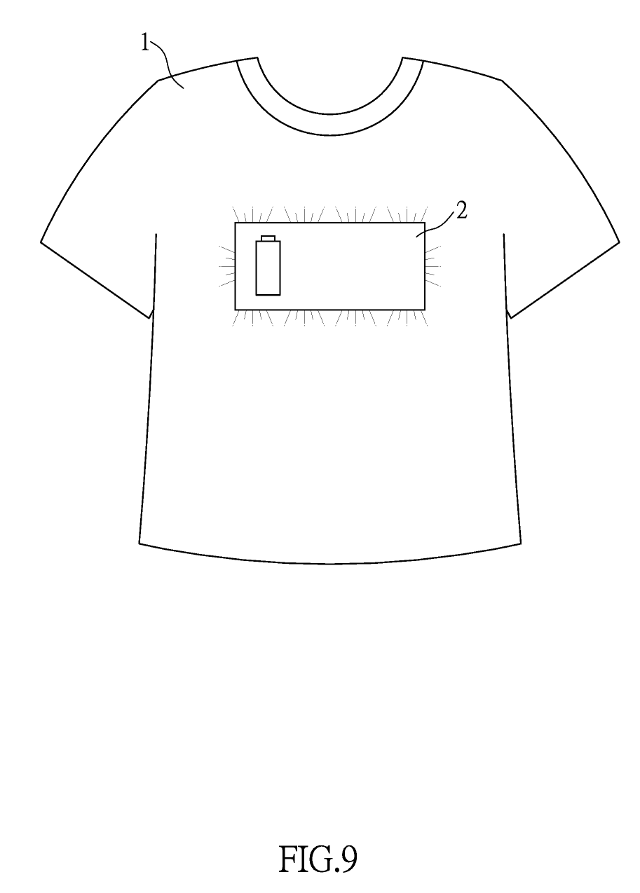

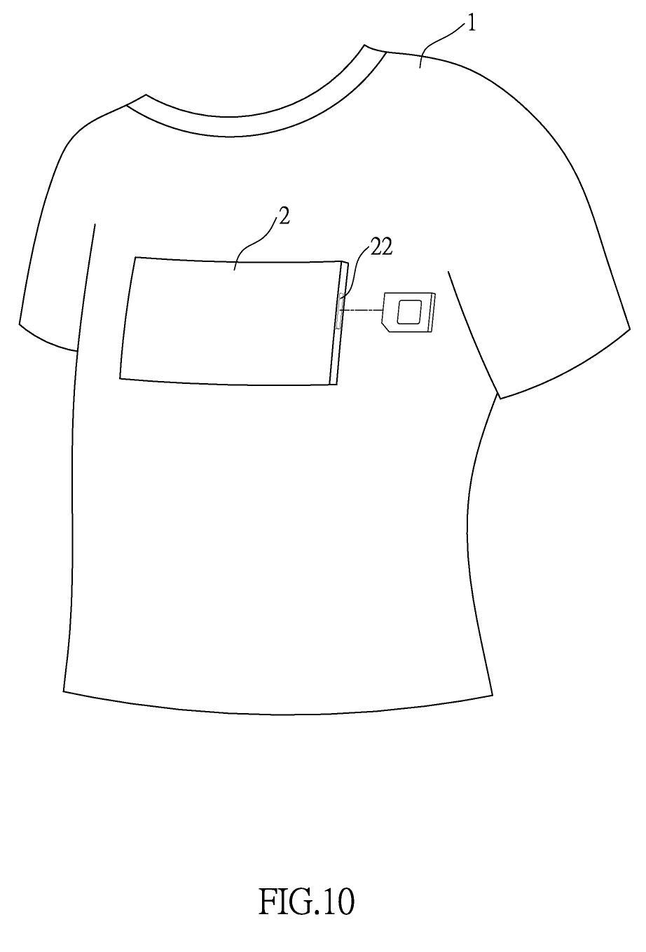

Please refer to FIGS. 7 to 10, the second embodiment of the present invention is illustrated and disclosed. The different between the second embodiment and the first embodiment is that the fixture 1 further includes at least one sewing portion 11 and two ends of at least one leas 5 are respectively and electrically connected to the display member 2 and the sensing member 3. It is important that the at least one lead 5 is buried in the at least one sewing portion 11. Generally, the position of sewing portion 11 of the clothes is arranged at shoulder, chest, and two sides of body. Therefore, because the lead 5 of the fixture 1 is buried in the sewing portion 11, it may make the fixture 1 neat and beautiful. The uncomfortability of the user may be improved so that the user moves and exercises smoothly. The sensing member 3 may effectively sense and the display member 3 may precisely show/display. The second embodiment may further include a reader 22 built therein. The reader 22 is mainly used to rad the data stored in the portable external hard driver, SIM card, or USB flash drive. The data read from the reader 22may be transmitted to the sensing member 3. In order to cooperate the data transmission, the sensing member 3 further include an access unit 34 for connecting the control unit 31. Therefore, the data read from the reader 22 may be transmitted to the access unit 34 of the sensing member 3 for being accessed so as to drive the control unit 31 to change modes according to the data. Otherwise, the data(or message) is transmitted to display member 2 by the transmission unit 32 built-in the sensing member 3 to provide others to know. For example, when the display member 2 is the flexible LCD, the reader 22 may read the word message/data to show/display on the flexible LCD. No matter what the underlying diseases or family history of diseases of the user are, the people around the user may know the user what the condition is by sowing the word message/data on the flexible LCD (shown as in FIG. 10, but not shown words). Please refer to FIG. 9, the control unit 31 may further include a power available mode 315. When the power in the power supply unit 33 of the sensing member 3 is not enough to provide to the display member 2 and the light emitting element 21, the power available mode 315 of the control unit 31 may control the display member 2 to show/display the power is not enough. When the display member 2 is the flexible LCD, a figure of low power is shown/displayed on the flexible LCD so as let the user know the power is low. When the display member 2 is the button or the light strip, the flash mode 311 may be applied to remind the user the power is low by flashing with one color or flashing with different colors. Therefore, the people around the user may know the power is low or not enough.

In conclusion, the field of the fixture 1 may be applied very widely. It is mainly applied to the field of emergency treatment/first aid so that the user, the people around the user, or the emergency care personnel may immediately know what happened and what the condition of the user is and then make the emergency treatment/first aid in time to prevent the accidents from happening. It may be also applied to the field of competitions for the recorder convenient to record and recognize so as to reduce manpower and prevent from cheating. The lead 5 is buried in the fixture 1 but not exposed so as to make it beautiful.

The foregoing descriptions are merely the exemplified embodiments of the present invention, where the scope of the claim of the present invention is not intended to be limited by the embodiments. Any equivalent embodiments or modifications without departing from the spirit and scope of the present invention are therefore intended to be embraced.

The disclosed structure of the invention has not appeared in the prior art and features efficacy better than the prior structure which is construed to be a novel and creative invention, thereby filing the present application herein subject to the patent law.

What is claimed is:

1. A motion sensing device, comprising:
a fixture, arranged with an user;
a display member, assembled to an outer surface of the fixture;
a sensing member, assembled to the fixture for sensing a physiological condition of the user, the sensing member includes a control unit and a transmission unit which are built therein, the control unit is electrically connected to the display member, and further the sensing member includes a power supply unit for driving the operation of the display member; and
a mobile member, electrically and wirelessly connected to the sensing member, the mobile member includes a receiving unit which is built therein, and the receiving unit is wirelessly connected to the transmission unit built-in the sensing member;
wherein the physiological condition of the user may be shown on the display member through the sensing member assembled to the fixture and cooperated with the display member, and then the physiological condition is wirelessly transmitted to the mobile member for providing to others or himself/herself to know anytime and anywhere.

2. The motion sensing device as claimed in claim 1, wherein the display member is a flexible liquid crystal display, a button, or a light strip.

3. The motion sensing device as claimed in claim 2, wherein the display member is the button and a light emitting element is arranged therein, the light emitting element is electrically connected to the sensing member, and the power supply unit is driving the light emitting element to illuminate.

4. The motion sensing device as claimed in claim 1, wherein the control unit is built-in a flash mode, an alarm mode, a safety mode, and a race mode.

5. The motion sensing device as claimed in claim 4, wherein the mobile member is a smart phone, a pad or tablet, or a transmitter, the connection of the mobile member and the sensing member is with cloud, bluetooth, infrared rays, or radio frequency.

6. The motion sensing device as claimed in claim 1 wherein the fixture is clothes, a waistband, a wristband, an armband, or a sports bra.

7. A motion sensing device, comprising:
a fixture, worn on an user and having at least one sewing portion;
a display member, assembled to an outer surface of the fixture;
a sensing member, assembled to the fixture for sensing a physiological condition of the user, the sensing member includes a control unit and a transmission unit which are built therein, the control unit is electrically connected to the display member, and further the sensing member includes a power supply unit for driving the operation of the display member; and
at least one lead is built in the at least one sewing portion, two ends of the at least one lead are respectively electrically connected to the display member and the sensing member;
wherein the physiological condition of the user may be shown on the display member through the sensing member assembled to the fixture and cooperated with the display member, and then the physiological condition is wirelessly transmitted to the mobile member for providing to others or himself/herself to know anytime and anywhere; and
wherein the at least one lead is built-in the at least one sewing portion so as to match a body curve of the user.

8. The motion sensing device as claimed in claim 7, wherein the display member is a flexible liquid crystal display, a button, or a light strip; the display member further includes a reader built therein, the reader is read a portable external hard driver, a SIM card, or an USB flash drive and transmitted data to the sensing member, the sensing member includes an access unit which is electrically connected to the control unit, and the access unit is accessed the data read from the reader.

9. The motion sensing device as claimed in claim 8, wherein the display member is the button and a light emitting element is arranged therein, the light emitting element is electrically connected to the sensing member, and the power supply unit is driving the light emitting element to illuminate.

10. The motion sensing device as claimed in claim 7, wherein the control unit is built-in a flash mode, an alarm mode, a safety mode, and a race mode.

11. The motion sensing device as claimed in claim 10, wherein the fixture is clothes, a girdle, a wristband, an armband, or a sports bra.
